(19) United States
(12) Patent Application Publication
(10) Pub. No.: US 2026/0144944 A1
(43) Pub. Date: May 28, 2026

(54) NEBULIZER MONITORING SYSTEM AND MEDICATION ADHERENCE MONITORING METHOD

(71) Applicant: HCMed Innovations Co., LTD., Taipei City (TW)

(72) Inventors: YI-HUNG WANG, New Taipei City (TW); SHIH-CHAO LUO, New Taipei City (TW); CHIEN-SHEN TSAI, New Taipei City (TW)

(21) Appl. No.: 19/192,284

(22) Filed: Apr. 28, 2025

Related U.S. Application Data
(60) Provisional application No. 63/639,875, filed on Apr. 29, 2024.

(30) Foreign Application Priority Data
Apr. 22, 2025 (CN) 202510506253.4

(51) Int. Cl. A61M 11/00

(52) U.S. Cl. CPC A61M 11/005; A61M 2202/0007; A61M 2205/3344; A61M 2205/3553; A61M 2205/502; A61M 2205/52; A61M 2205/581; A61M 2205/582; A61M 2205/583; A61M 2205/6009; A61M 2205/6072; A61M 2205/8206; A61M 2210/0625; A61M 2230/005; A61M 2230/40

(57) ABSTRACT

A nebulizer monitoring system and a medication adherence monitoring method. The nebulizer monitoring system includes a nebulizer and a remote monitoring platform. The nebulizer has a mouthpiece. The nebulizer includes a nebulizing module, a sensing assembly, and a control module. The control module is electrically connected to the nebulizing module and the sensing assembly. The remote monitoring platform includes a computing module. The computing module is configured to analyze and calculate at least two adherence parameters and generate a breathing pattern. The control module is configured to adjust a nebulizing frequency and an aerosol volume of the nebulizing module based on the breathing pattern.

### CROSS-REFERENCE TO RELATED PATENT APPLICATION

This application claims the benefit of priority to China Patent Application No. 202510506253.4, filed on Apr. 22, 2025, in the People's Republic of China. The entire content of the above identified application is incorporated herein by reference.

This application claims the benefit of priority to the U.S. Provisional Patent Application Ser. No. 63/639,875, filed on Apr. 29, 2024, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a monitoring system and a monitoring method, and more particularly to a nebulizer monitoring system and a medication adherence monitoring method applied to the nebulizer monitoring system.

### BACKGROUND OF THE DISCLOSURE

A nebulizer is primarily used to nebulize liquid medication into fine droplets, enabling the patient to directly inhale the medication into the lungs for rapid therapeutic effect. When using the nebulizer, the patient needs to maintain slow and deep breathing to ensure that the medication properly reaches the lungs and achieves maximum effectiveness.

Existing nebulizers generally only have basic data recording functions, such as the number of administrations, the timing of medication use, and the dosage, for subsequent review by the user. In other words, existing nebulizers are incapable of real-time detection of the user's breathing condition, and thus cannot confirm whether the user maintains slow and deep breathing during the treatment process to ensure proper absorption of the medication. Furthermore, existing nebulizers are unable to provide real-time feedback or adjust its mode of operation according to the user's breathing pattern. Therefore, the medication adherence (i.e., the extent to which the user follows medical advice regarding medication timing, dosage, and frequency) cannot be improved, and treatment outcomes may be negatively impacted.

Therefore, how to improve the monitoring and feedback mechanism of the nebulizer and overcome the above-mentioned problems has become an important issue to be addressed in the relevant art.

### SUMMARY OF THE DISCLOSURE

In response to the above-referenced technical inadequacies, the present disclosure provides a nebulizer monitoring system and a medication adherence monitoring method applied to the nebulizer monitoring system.

In order to solve the above-mentioned problems, one of the technical aspects adopted by the present disclosure is to provide a nebulizer monitoring system, which includes a nebulizer and a remote monitoring platform. The nebulizer has a mouthpiece. The nebulizer includes a nebulizing module, a sensing assembly, and a control module. The control module is electrically connected to the nebulizing module and the sensing assembly. The sensing assembly is configured to detect at least two adherence parameters generated by the nebulizer when a user inhales and exhales through the mouthpiece. The remote monitoring platform is in signal communication with the nebulizer. The remote monitoring platform includes a computing module, and the computing module is configured to analyze and calculate the at least two adherence parameters and generate a breathing pattern. The control module outputs a drive signal to the nebulizing module based on the breathing pattern, so as to adjust a nebulizing frequency and an aerosol volume of the nebulizing module.

In order to solve the above-mentioned problems, another one of the technical aspects adopted by the present disclosure is to provide a medication adherence monitoring method applied to the nebulizer monitoring system. The monitoring method includes: providing the nebulizer to a user, and detecting at least two adherence parameters through the sensing assembly; analyzing and calculating the at least two adherence parameters detected by the sensing assembly through the computing module to generate a breathing pattern; and outputting a drive signal to the nebulizing module through the control module based on the breathing pattern, so as to adjust a nebulizing frequency and an aerosol volume of the nebulizing module.

Therefore, in the nebulizer monitoring system and medication adherence monitoring method provided by the present disclosure, the user's breathing condition can be detected in real time, and the at least two adherence parameters can be analyzed and obtained by the computing module to output an optimized breathing pattern. Then, based on this optimized breathing pattern, the control module dynamically adjusts the nebulizing frequency and the aerosol volume of the nebulizing module. The nebulizer can actively modulate the aerosol volume in accordance with the user's breathing rhythm. At the same time, the feedback module can guide the user to maintain a slow and deep breathing pattern, thereby enhancing drug absorption efficiency, optimizing therapeutic outcomes, and improving medication adherence.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a nebulizer monitoring system according to the present disclosure;

FIG. 2 is a schematic view of a user operating a nebulizer according to the present disclosure;

FIG. 3 is a functional block diagram of the nebulizer monitoring system according to the present disclosure;

FIG. 4 is a schematic view of a remote monitoring platform of the nebulizer monitoring system being remotely connected to a mobile device according to the present disclosure; and

FIG. 5 is a schematic view of the nebulizer of the nebulizer monitoring system identifying a specific medication according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### Embodiment

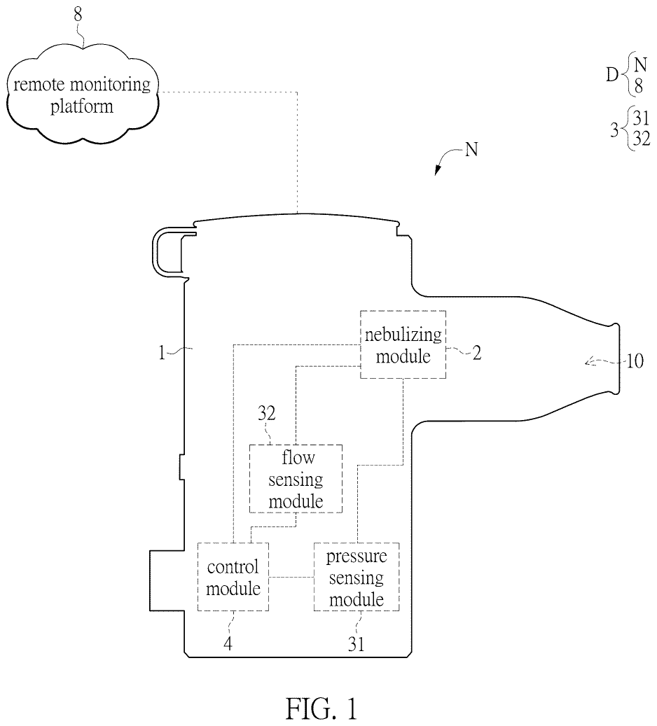

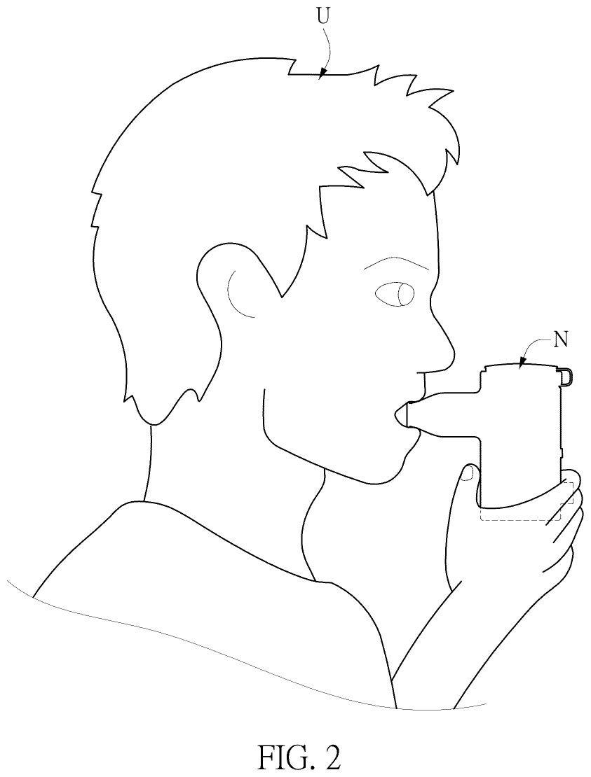

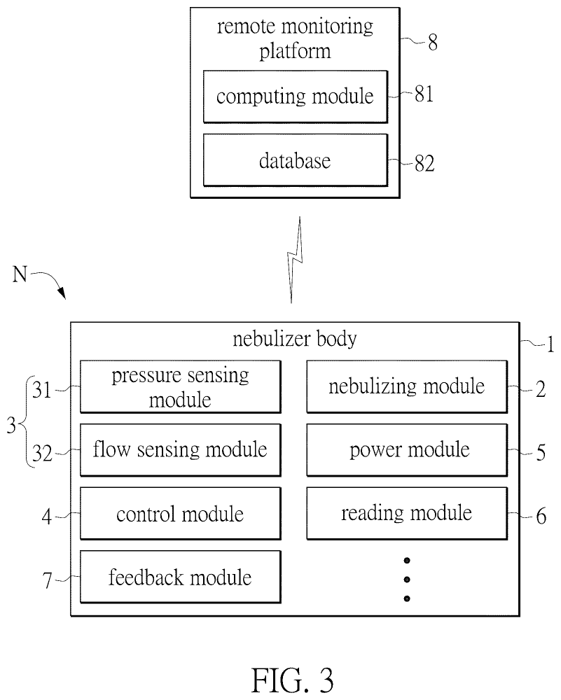

Reference is made to FIGS. 1 to 3. FIG. 1 is a schematic view of a nebulizer monitoring system according to the present disclosure, FIG. 2 is a schematic view of a user operating a nebulizer according to the present disclosure, and FIG. 3 is a functional block diagram of the nebulizer monitoring system according to the present disclosure. The present disclosure provides a nebulizer monitoring system D, which enables real-time monitoring of a user's medication adherence while using a nebulizer. The nebulizer monitoring system D includes a nebulizer N and a remote monitoring platform 8. The remote monitoring platform 8 is in signal communication with the nebulizer N. Specifically, the remote monitoring platform 8 can be a remote server, and the nebulizer N can be internally equipped with a wireless communication module (not shown in the figures) that is in signal communication with the remote monitoring platform 8 for remote data transmission and monitoring. For example, the wireless communication module may include communication components such as Wi-Fi®, Bluetooth®, or LTE, but the present disclosure is not limited thereto.

The nebulizer N includes a nebulizer body 1, a nebulizing module 2, a sensing assembly 3, and a control module 4. The control module 4 is electrically connected to the nebulizing module 2 and the sensing assembly 3. The nebulizer body 1 is configured to store liquid medication, and the nebulizer body 1 includes a mouthpiece 10 through which a user U inhales the medication mist. The nebulizing module 2, the sensing assembly 3, and the control module 4 are disposed within the nebulizer body 1. In one embodiment, the sensing assembly 3 is disposed adjacent to the mouthpiece 10, but the present disclosure is not limited thereto. For example, the nebulizing module 2 can be an ultrasonic nebulizer or mesh-type ultrasonic nebulizer made of piezoelectric material that nebulizes the liquid medication through high-frequency ultrasonic vibration. The sensing assembly 3 includes a pressure sensing module 31, such as a MEMS pressure sensor. The control module 4, such as a microcontroller, is configured to receive data from the pressure sensing module 31 and dynamically adjust a nebulizing frequency and an aerosol volume of the nebulizing module 2.

Furthermore, the user U can perform a method for monitoring medication adherence using the nebulizer monitoring system D. The monitoring method includes at least the following steps S1 to S3:
Step S1: providing the nebulizer N to the user U and detecting at least two adherence parameters through the sensing assembly 3.

Step S2: analyzing and calculating the at least two adherence parameters detected by the sensing assembly 3 through a computing module 81 of the remote monitoring platform 8 to generate a breathing pattern.

Step S3: outputting a drive signal from the control module 4 to the nebulizing module 2 based on the breathing pattern, so as to adjust the nebulizing frequency and the aerosol volume of the nebulizing module 2.

The at least two adherence parameters include at least two of a pressure variation parameter, an inhalation time parameter, an inhalation flow parameter, an exhalation flow parameter, a power status parameter, and an identification code. The adherence parameters of the present disclosure refer to parameters that are used to determine whether the user follows the physician's prescription and the pharmacist's medication instructions during nebulized therapy. Accordingly, by combining at least two of the pressure variation parameter, the inhalation time parameter, the inhalation flow parameter, the exhalation flow parameter, the power status parameter, and the identification code, the treatment condition of the user using the nebulizer can be recorded, allowing the degree of medication adherence to be determined.

Specifically, in one embodiment, when the user U operates the nebulizer N, the pressure sensing module 31 is configured to detect pressure variations at the mouthpiece 10 during breathing and to obtain the pressure variation parameters. Meanwhile, the pressure variation parameters can also be used in conjunction with a built-in timer (not shown in figures) to obtain the inhalation time parameters. The remote monitoring platform 8 includes the computing module 81, which is configured to receive, analyze, and process data from the nebulizer N to provide real-time monitoring and intelligent control. For example, the computing module 81 can be a processor. The computing module 81 is configured to analyze and calculate the pressure variation parameters detected by the pressure sensing module 31 and the inhalation time parameters, and to record and evaluate the user's medication usage condition. Based on the pressure variation and inhalation time parameters, the computing module 81 calculates and generates an optimized breathing pattern.

Subsequently, the control module 4 outputs a drive signal to the nebulizing module 2 according to the optimized breathing pattern, enabling the nebulizing module 2 to instantly adjust the nebulizing frequency and the aerosol volume, thereby controlling the concentration and rate of the released aerosol to match the inhalation duration and intensity of the user U. Through the dynamic control mechanism established by integrating the control module 4 and the nebulizing module 2, the user's drug absorption efficiency can be optimized during aerosol therapy, thereby further enhancing the therapeutic outcomes.

As shown in FIG. 1, the sensing assembly 3 further includes a flow sensing module 32, which is disposed within the nebulizer body 1. Preferably, the flow sensing module 32 is adjacent to the mouthpiece 10, but the present disclosure is not limited thereto. For example, the flow sensing module 32 can be a hot wire flowmeter or a mechanical flow sensor. The flow sensing module 32 is configured to detect changes in airflow at the mouthpiece 10 (i.e., inhalation flow parameters and exhalation flow parameters) during the user's breathing with the nebulizer N, and to determine the inhalation and exhalation states and the breathing intensity of the user U based on airflow speed and direction. For instance, when the flow sensing module 32 detects airflow exiting the nebulizer body 1 (i.e., the user U inhales), the nebulizing module 2 is activated and releases the medication mist. On the other hand, when the flow sensing module 32 detects airflow entering the nebulizer body 1 (i.e., the user U exhales or ceases inhalation), the nebulizing module 2 stops nebulization.

Through the configuration of the flow sensing module 32 in combination with the pressure sensing module 31, the computing module 81 not only analyzes and calculates the pressure variation parameters detected by the pressure sensing module 31 during breathing, but also analyzes and calculates the inhalation flow and exhalation flow parameters detected by the flow sensing module 32, thereby further optimizing the breathing pattern. Based on the optimized breathing pattern, the control module 4 can control the nebulizing module 2 to instantly adjust the nebulizing frequency and further control the concentration and rate of the released aerosol to match the inhalation duration and intensity of user U.

Furthermore, the pressure sensing module 31 and the flow sensing module 32 are capable of detecting airflow pressure variations and airflow volume/rate during the user's aerosol therapy to determine whether factors such as rapid breathing affect drug absorption, whether the user's inhalation is smooth and stable, and whether the user maintains a slow and deep breathing pattern. Based on aforementioned real-time data, the control module 4 can dynamically adjust the nebulizing frequency and the aerosol volume of the nebulizer N, ensuring that the nebulizer N provides the appropriate drug delivery mode during the user's inhalation. In this way, by interpreting at least two adherence parameters from among the pressure variation parameter, the inhalation time parameter, the inhalation flow parameter, and the exhalation flow parameter, the remote monitoring platform 8 can determine the user's medication adherence to achieve the intended monitoring purpose.

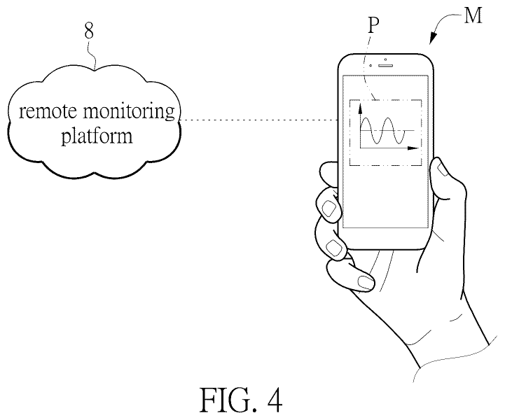

The remote monitoring platform 8 further includes a database 82, which is in signal communication with the nebulizing module 2, the pressure sensing module 31, the flow sensing module 32, the control module 4, and the computing module 81. For example, the database 82 can be a system used for storing, managing, and retrieving data, and the database 82 is used to store various monitoring data from the nebulizer N, such as the drive signal, the pressure variation parameters, the inhalation time parameters, the inhalation flow parameters, the exhalation flow parameters, and the power status parameters. Reference is made to FIG. 4, which is a schematic view of a remote monitoring platform of the nebulizer monitoring system being remotely connected to a mobile device according to the present disclosure. The remote monitoring platform 8 can be remotely connected to a mobile device M (e.g., a smartphone, a tablet computer, or a laptop). The computing module 81 can display a breathing waveform P corresponding to the breathing pattern in an application executed on the mobile device M.

Specifically, the remote monitoring platform 8 can connect remotely to the mobile device M, thereby allowing the user U or medical personnel to view relevant data and treatment progress in real time. The computing module 81 can convert the calculated breathing pattern into the breathing waveform P, which is displayed in the application executed on the mobile device M. For example, the breathing waveform P specifically shows the user's breathing intensity, a ratio of inhalation to exhalation time, and other parameters such as respiratory rate. Therefore, the user's breathing status can be intuitively presented to help the user U determine whether their current breathing condition aligns with the optimal mode of treatment (i.e., the optimized breathing pattern), thereby enhancing medication adherence.

Referring also to FIG. 3, the nebulizer N further includes a power module 5. The power module 5 is disposed within the nebulizer body 1 and supplies electrical power to the nebulizing module 2, the pressure sensing module 31, the flow sensing module 32, and the control module 4. The power module 5 can be, for example, a rechargeable battery or a replaceable disposable battery. In addition, the power module 5 can be in signal communication with the remote monitoring platform 8 and store relevant power status parameters in the database 82. For example, the power status parameters can include the number of power-on events, power-off events, and hibernation events of the nebulizer N, battery level trends, and charging cycles, for the purpose of remote monitoring and maintenance.

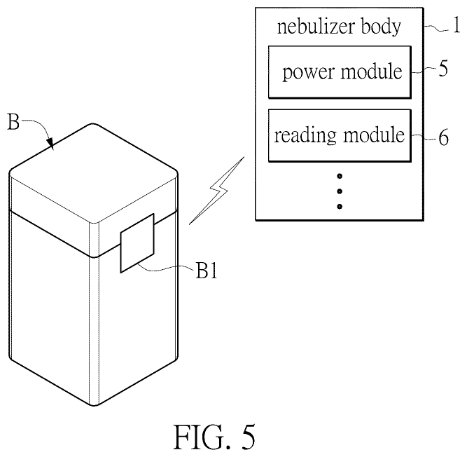

Reference is made to FIGS. 3 and 5. FIG. 5 is a schematic view of the nebulizer of the nebulizer monitoring system identifying a specific medication according to the present disclosure. The nebulizer N is designed to be used with a specific medication, and a container B of the medication is provided with an identification code B1. To ensure that the nebulizer N operates only with the designated medication, the nebulizer N is equipped with a reading module 6 for reading the identification code B1. When the reading module 6 successfully recognizes the identification code B1, the control module 4 outputs an activation signal to the power module 5, enabling the power module 5 to supply electrical power and activate the nebulizer N, thereby ensuring that the nebulizer N operates only after the medication has been verified.

Furthermore, the database 82 can further be used to store information related to the identification code B1 for medication management and device monitoring. For example, the identification code B1 includes an NFC tag, a two-dimensional matrix barcode (e.g., QR code), an RFID tag, or other authentication mechanisms to ensure medication accuracy and traceability. Accordingly, the reading module 6 can include an NFC reader, a barcode scanner, or an RFID reader, or other sensing devices suitable for various medication identification methods.

Since the power status parameters and the identification code can further be used in conjunction with the pressure variation parameters, the inhalation time parameters, the inhalation flow parameters, and the exhalation flow parameters to determine the user's medication adherence, the power status parameters and the identification code can be defined as adherence parameters and serve as indicators for assessing the user's medication adherence.

The nebulizer N further includes a feedback module 7, which is electrically connected to the control module 4 and provides real-time feedback to help the user U follow the correct breathing pattern during treatment. When the user U uses the nebulizer N according to the breathing pattern, the feedback module 7 generates a corresponding sensing signal to guide or remind the user U. For example, the feedback module 7 can include a vibration element (e.g., a micro vibration motor), an audio element (e.g., a built-in buzzer), and a visual element (e.g., an LED indicator or display screen). The sensing signal includes at least one of a vibration signal provided by the micro vibration motor, an audio signal provided by the buzzer, and a visual signal provided by the LED indicator or display screen. These sensing signals can be used individually or in combination to enhance the user's experience. Additionally, these sensing signals can guide the user U to maintain slow and deep breathing to ensure sufficient drug inhalation. Through the provision of the feedback module 7, the user U can experience significantly improved therapeutic effects, thereby increasing their medication adherence.

### Beneficial Effects of the Embodiment

The nebulizer monitoring system D provided by the present disclosure, together with the medication adherence monitoring method applied to the monitoring system, enables the computing module 81 to analyze at least two adherence parameters detected by the sensing assembly 3 for monitoring the user's medication adherence, and to further calculate and output an optimized breathing pattern. For example, the computing module 81 can analyze at least two adherence parameters, such as the pressure variation parameters, the inhalation time parameters, the inhalation flow parameters, and the exhalation flow parameters that are obtained by the pressure sensing module 31 and the flow sensing module 32, to monitor the user's medication adherence. At the same time, the control module 4 can detect the user's breathing condition in real time to provide an optimized breathing pattern. Based on the optimized breathing pattern, the control module 4 dynamically adjusts the nebulizing frequency and the aerosol volume of the nebulizing module 2, thereby enhancing the user's drug absorption efficiency during treatment and optimizing therapeutic outcomes.

Furthermore, through the configuration of the flow sensing module 32 in combination with the pressure sensing module 31, the computing module 81 not only analyzes and calculates the pressure variation parameters detected by the pressure sensing module 31 during breathing, but also analyzes and calculates the inhalation flow and exhalation flow parameters detected by the flow sensing module 32, thereby further optimizing the breathing pattern. Based on the optimized breathing pattern, the control module 4 can control the nebulizing module 2 to instantly adjust the nebulizing frequency and further control the concentration and rate of the released aerosol to match the inhalation duration and intensity of the user U. Moreover, through the configuration of the feedback module 7, the user U can be guided to maintain a slow and deep breathing rhythm to ensure proper drug inhalation, thereby further improving the user's medication adherence.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A nebulizer monitoring system, comprising:
a nebulizer having a mouthpiece, wherein the nebulizer includes a nebulizing module, a sensing assembly, and a control module, the control module is electrically connected to the nebulizing module and the sensing assembly, the sensing assembly is configured to detect at least two adherence parameters generated by the nebulizer when a user inhales and exhales through the mouthpiece; and
a remote monitoring platform being in signal communication with the nebulizer, wherein the remote monitoring platform includes a computing module, the computing module is configured to analyze and calculate the at least two adherence parameters and generate a breathing pattern, and the control module outputs a drive signal to the nebulizing module based on the breathing pattern, so as to adjust a nebulizing frequency and an aerosol volume of the nebulizing module.

2. The nebulizer monitoring system according to claim 1, wherein the at least two adherence parameters include at least two of a pressure variation parameter, an inhalation time parameter, an inhalation flow parameter, an exhalation flow parameter, a power status parameter, and an identification code.

3. The nebulizer monitoring system according to claim 2, wherein the remote monitoring platform is configured to be remotely connected to a mobile device, and the computing module is configured to display a breathing waveform corresponding to the breathing pattern in an application executed on the mobile device.

4. The nebulizer monitoring system according to claim 2, wherein the remote monitoring platform further includes a database, the database is configured to be in signal communication with the nebulizing module, the sensing assembly, the control module, and the computing module, and the database is further configured to store the at least two adherence parameters.

5. The nebulizer monitoring system according to claim 4, wherein the nebulizer further includes a power module that is configured to supply power to the sensing assembly, the nebulizing module, and the control module, the power module is in signal communication with the remote monitoring platform, the database is configured to store the power status parameters of the nebulizer; and wherein the power status parameters include numbers of power-on events, power-off events, and hibernation events of the nebulizer.

6. The nebulizer monitoring system according to claim 4, wherein the nebulizer is configured for use with a specific medication, a packaging of the specific medication is provided with the identification code, the nebulizer further includes a reading module corresponding to the identification code, the nebulizer is activated by reading the identification code through the reading module, and the database is provided for storing information associated with the identification code.

7. The nebulizer monitoring system according to claim 6, wherein the identification code includes an NFC tag, a two-dimensional matrix barcode, or an RFID tag, and the reading module includes an NFC reader, a barcode scanner, or an RFID reader.

8. The nebulizer monitoring system according to claim 2, wherein the sensing assembly includes a pressure sensing module, and the pressure sensing module is configured to detect the pressure variation parameters when the user inhales and exhales through the nebulizer.

9. The nebulizer monitoring system according to claim 8, wherein the sensing assembly further includes a flow sensing module, and the flow sensing module is configured to detect the inhalation flow parameters and the exhalation flow parameters when the user inhales and exhales through the nebulizer.

10. The nebulizer monitoring system according to claim 1, wherein the nebulizer further includes a feedback module, the feedback module is electrically connected to the control module; and wherein, when the user operates the nebulizer in accordance with the breathing pattern, the feedback module is configured to generate a sensing signal correspondingly.

11. The nebulizer monitoring system according to claim 10, wherein the sensing signal includes at least one of a vibration signal, an audio signal, and a visual signal.

12. A medication adherence monitoring method applied to a nebulizer monitoring system, the nebulizer monitoring system including a nebulizer and a remote monitoring platform, the nebulizer having a mouthpiece, the nebulizer including a nebulizing module, a sensing assembly, and a control module, the control module being electrically connected to the nebulizing module and the sensing assembly, and the remote monitoring platform including a computing module, the medication adherence monitoring method comprising:
providing the nebulizer to a user, and detecting at least two adherence parameters through the sensing assembly;
analyzing and calculating the at least two adherence parameters detected by the sensing assembly through the computing module to generate a breathing pattern; and
outputting a drive signal to the nebulizing module through the control module based on the breathing pattern, so as to adjust a nebulizing frequency and an aerosol volume of the nebulizing module.

13. The medication adherence monitoring method according to claim 12, wherein the at least two adherence parameters include at least two of a pressure variation parameter, an inhalation time parameter, an inhalation flow parameter, an exhalation flow parameter, a power status parameter, and an identification code.

14. The medication adherence monitoring method according to claim 13, wherein the remote monitoring platform is configured to be remotely connected to a mobile device, and the computing module is configured to display a breathing waveform corresponding to the breathing pattern in an application on the mobile device.

15. The medication adherence monitoring method according to claim 13, wherein the remote monitoring platform further includes a database, the database is configured to be in signal communication with the nebulizing module, the sensing assembly, the control module, and the computing module, and the database is further configured to store the at least two adherence parameters.

16. The medication adherence monitoring method according to claim 15, wherein the nebulizer further includes a power module that is configured to supply electrical power to the sensing assembly, the nebulizing module, and the control module, the power module is in signal communication with the remote monitoring platform, the database is configured to store the power status parameters of the nebulizer; and wherein the power status parameters include numbers of power-on events, power-off events, and hibernation events of the nebulizer.

17. The medication adherence monitoring method according to claim 15, wherein the nebulizer is configured for use with a specific medication, a packaging of the specific medication is provided with an identification code, the nebulizer further includes a reading module corresponding to the identification code, the nebulizer is activated by reading the identification code through the reading module, and the database is provided for storing information associated with the identification code.

18. The medication adherence monitoring method according to claim 17, wherein the identification code includes an NFC tag, a two-dimensional matrix barcode, or an RFID tag, and the reading module includes an NFC reader, a barcode scanner, or an RFID reader.

19. The medication adherence monitoring method according to claim 13, wherein the sensing assembly includes a pressure sensing module, and the pressure sensing module is configured to detect the pressure variation parameters when the user inhales and exhales through the nebulizer.

20. The medication adherence monitoring method according to claim 19, wherein the sensing assembly further includes a flow sensing module, and the flow sensing module is configured to detect the inhalation flow parameters and the exhalation flow parameters when the user inhales and exhales through the nebulizer.

21. The medication adherence monitoring method according to claim 13, wherein the nebulizer further includes a feedback module, the feedback module is electrically connected to the control module; and wherein, when the user operates the nebulizer in accordance with the breathing pattern, the feedback module is configured to generate a sensing signal correspondingly.

22. The medication adherence monitoring method according to claim 21, wherein the sensing signal includes at least one of a vibration signal, an audio signal, and a visual signal.
